(19) United States
(12) Patent Application Publication
(10) Pub. No.: US 2025/0228436 A1
(43) Pub. Date: Jul. 17, 2025

(54) MEDICAL SYSTEMS AND METHODS FOR MONITORING LIMITED USE COMPONENTS

(71) Applicant: Boston Scientific Scimed, Inc., Maple Grove, MN (US)

(72) Inventor: Kirsten VIERING, Newton, MA (US)

(73) Assignee: Boston Scientific Scimed, Inc., Maple Grove, MN (US)

(21) Appl. No.: 19/010,499

(22) Filed: Jan. 6, 2025

Related U.S. Application Data
(60) Provisional application No. 63/620,234, filed on Jan. 12, 2024.

(51) Int. Cl. A61B 1/00; A61B 90/00

(52) U.S. Cl. CPC A61B 1/00124; A61B 1/00018; A61B 1/0002; A61B 1/00055; A61B 1/00059; A61B 1/00062; A61B 90/08; A61B 2090/0803; A61B 2090/0814

(57) ABSTRACT

A medical system includes a computing system, and a connection device having a lifetime and including first and second connectors connectable to the computing system and one or more medical devices, respectively. The computing system is configured to perform operations to independently monitor usage of the first and second connectors, including: detecting a connection of the first and second connectors to the computing system and a first medical device, respectively; receiving usage information of the first and second connectors; updating the usage information based on the connection of the first and second connectors to the computing system and the first medical device, respectively; and in response to determining at least one of a plurality of predetermined thresholds specific to each of the first and second connectors and indicating the lifetime of the connection device is approaching or has been reached, has been met or exceeded, performing one or more actions.

### CROSS-REFERENCE TO RELATED APPLICATIONS

This application claims the benefit of priority to U.S. Provisional Application No. 63/620,234, filed on Jan. 12, 2024, which is incorporated by reference herein in its entirety.

### TECHNICAL FIELD

The disclosure relates generally to medical systems and methods for monitoring limited use components. More specifically, aspects of the disclosure pertain to medical systems and methods for monitoring usage of a connection device configured to removably connect a computing system and a medical device.

### BACKGROUND

A medical system may include a connection device for removably connecting a medical device to a computing system to perform a medical procedure. The medical device may be single use or disposable to minimize cross-contamination between patients. For example, after the medical procedure is performed, the medical device may be disconnected from the computing system and disposed of. A new medical device may then be used for a next procedure. Disposing of a medical device after every procedure creates large amounts of waste and higher costs.

### SUMMARY

Aspects of techniques described herein relate to a medical system. An example medical system includes a computing system, and a connection device configured to removably connect one or more medical devices to the computing system for use in one or more medical procedures. The connection device includes a memory, a first connector connectable to the computing system, and a second connector, different from the first connector, connectable to the one or more medical devices. The connection device has a lifetime for use. The computing system includes at least one memory configured to store instructions and at least one processor configured to execute the instructions to perform operations to independently monitor usage of the first connector and the second connector of the connection device. The operations include: detecting a connection of the first connector to the computing system; determining a connection of the second connector to a first medical device of the one or more medical devices based on one or more data interactions detected between the computing system and the first medical device; receiving usage information of the connection device, including usage information specific to each of the first connector and the second connector; updating the usage information for each of the first connector and the second connector based on the connection of the first connector to the computing system and the connection of the second connector to the first medical device, respectively; determining whether at least one of a plurality of predetermined thresholds indicating the lifetime of the connection device is approaching or has been reached has been met or exceeded based on the received usage information or the updated usage information, the plurality of predetermined thresholds including one or more predetermined thresholds specific to each of the first connector and the second connector; and in response to determining at least one of the plurality of predetermined thresholds has been met or exceeded, performing one or more actions to indicate the lifetime of the connection device is approaching or has been reached.

In any of the example medical systems disclosed herein, the operations may further include: in response to the connection of the first connector to the computing system, receiving an identifier associated with the connection device stored in the memory of the connection device, wherein the identifier is used to receive one or more of the usage information of the connection device or the plurality of predetermined thresholds. The received usage information and the updated usage information may be stored at least one of: locally by the computing system, locally in the memory of the connection device, or remotely by a data storage system. The received usage information may include usage information of the connection device previously monitored by the computing system or one or more other computing systems. The received usage information may include one or more of a number of connections, a total amount of usage time, or an amount of time elapsed from an earliest connection associated with each of the first connector and the second connector. The plurality of predetermined thresholds may include one or more of a threshold number of connections, a threshold amount of usage time, or a threshold amount of time elapsed from an earliest connection for each of the first connector and the second connector.

In some aspects, for the first connector, updating the usage information may include, in response to detecting the connection of the first connector to the computing system: incrementing a number of connections for the first connector; and initiating a first duration counter configured to measure an amount of time the first connector is connected to the computing system, wherein at predetermined intervals, the amount of time indicated by the first duration counter is used to update a total amount of usage time for the first connector. For the second connector, updating the usage information may include, in response to detecting the one or more data interactions between the computing system and the first medical device: incrementing a number of connections for the second connector; and initiating a second duration counter configured to measure an amount of time the one or more data interactions continue to occur between the computing system and the first medical device, wherein at predetermined intervals, the amount of time indicated by the second duration counter is used to update a total amount of usage time for the second connector.

In other aspects, the operations may further include: as the first connector remains connected to the computing system, determining a disconnection of the second connector from the first medical device based on a cessation of the one or more data interactions between the computing system and the first medical device; determining a connection of the second connector to a second medical device of the one or more medical devices based on one or more data interactions detected between the computing system and the second medical device; and updating the usage information for the second connector further based on the connection of the second connector to the second medical device.

In some examples, performing the one or more actions may include; generating a notification indicating the lifetime of the connection device is approaching or has been reached; and providing the notification for display on a display device associated with the computing system. In other examples, performing the one or more actions may include performing one or more actions to render the connection device useless. In some aspects, performing the one or more actions to render the connection device useless may include preventing a processing and display of data received from the first medical device via the connection device. In other aspects, the connection device further includes one or more electronic components that, when in a closed circuit state, serve as pass-through components to enable data flow between the computing system and the first medical device via the connection device, and performing the one or more actions to render the connection device useless includes electrically disabling the connection device by generating and transmitting a trigger signal to the connection device, wherein the trigger signal causes the one or more electronic components of the connection device to be switched to an open circuit state that prevents any data flow.

In some examples, the operations may further include: generating a user interface that includes an indicator of an amount of lifetime remaining for the connection device based on the updated usage information; and providing the user interface for display on a display device associated with the computing system. In other examples, the medical system is an endoscopic imaging system, and the connection device is a reusable umbilicus for removably connecting one or more endoscopes to the computing system.

Additionally, the techniques described herein relate to a computing system that is removably connectable to a medical device for use during a medical procedure via a connection device. An example computing system includes at least one memory configured to store instructions, and at least one processor configured to execute the instructions to perform operations to independently monitor usage of a first connector and a second connector of the connection device. The operations including: detecting a connection of the connection device to the computing system via the first connector; receiving an identifier associated with the connection device from the connection device; using the identifier, receiving: usage information of the connection device associated with at least a number of connections and a duration of use specific to each of the first connector and the second connector; and a plurality of predetermined thresholds associated with at least a number of connections and a duration of use that (i) indicate a lifetime of the connection device is approaching or has been reached and (ii) are specific to each of the first connector and the second connector; determining a connection of the second connector to the medical device based on one or more data interactions detected between the computing system and the medical device; determining whether at least one of the plurality of predetermined thresholds has been met or exceeded based on the received usage information; and in response to determining at least one of the plurality of predetermined thresholds has been met or exceeded, generating and providing for display a notification indicating the lifetime of the connection device is approaching or has been reached.

In any of the exemplary computing systems disclosed herein, in response to determining at least one of the plurality of predetermined thresholds has been met or exceeded, the operations may further include performing one or more actions to render the connection device useless. Also, the operations may further include updating the usage information for each of the first connector and the second connector based on the connection of the first connector to the computing system and the connection of the second connector to the medical device, respectively. Updating the usage information may include: for the first connector, in response to detecting the connection of the first connector to the computing system: incrementing a number of connections for the first connector; and initiating a first duration counter configured to measure an amount of time the first connector is connected to the computing system, wherein at predetermined intervals, the amount of time indicated by the first duration counter is used to update a total amount of usage time for the first connector; and for the second connector, in response to detecting the one or more data interactions between the computing system and the medical device: incrementing a number of connections for the second connector; and initiating a second duration counter configured to measure an amount of time the one or more data interactions continue to occur between computing system and the medical device, wherein at predetermined intervals, the amount of time indicated by the second duration counter is used to update a total amount of usage time for the second connector.

Other aspects of techniques described herein may relate to a method performed by a computing system that is removably connectable to a medical device for use during a medical procedure via a connection device including a first connector and a second connector. An example method includes: detecting a connection of the first connector of the connection device to the computing system; receiving an identifier associated with the connection device from the connection device; using the identifier, receiving: usage information of the connection device specific to each of the first connector and the second connector; and a plurality of predetermined thresholds indicating a lifetime of the connection device is approaching or has been reached, including one or more predetermined thresholds specific to each of the first connector and the second connector; updating the usage information for the first connector based on the detected connection and a usage monitoring of the first connector as the first connector remains connected to the computing system; determining a connection of the second connector of the connection device to the medical device based one or more data interactions between the computing system and the medical device; independently from the first connector, updating the usage information for the second connector based on the determined connection and a usage monitoring of the second connector as the second connector remains connected to the computing system; periodically throughout a duration of the connection of at least the first connector to the computing system, determining whether at least one of the plurality of predetermined thresholds has been met or exceeded based on the received usage information or the updated usage information; and in response to determining at least one of the plurality of predetermined thresholds has been met or exceeded, performing one or more actions to indicate the lifetime of the connection device is approaching or has been reached, including at least generating and providing for display a notification indicating the lifetime of the connection device is approaching or has been reached.

In any of the exemplary methods disclosed herein, performing the one or more actions may further include performing one or more actions to render the connection device useless.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 depicts an example environment in which a usage monitoring process for a limited use component of a medical system may be implemented.

FIG. 2A depicts an example endoscopic imaging system.

FIG. 2B depicts an example umbilicus of the endoscopic imaging system of FIG. 2A.

FIG. 3 depicts an example process for monitoring usage of a limited use component of a medical system.

FIGS. 4A-4D depict example notifications.

FIG. 5 depicts an example computing device.

### DETAILED DESCRIPTION

As briefly mentioned above, a medical device that is removably connectable to a computing system via a connection device to perform a medical procedure may be a single use or disposable device. Therefore, to help reduce waste and improve cost efficiency, the connection device may be reusable. For example, the connection device may be cleaned or sterilized between each medical procedure. However, the reusable connection device may have a limited lifetime given that the reusable connection device may have components prone to wear and tear or breakage after a certain number of uses, which may be further impacted by sterilization and/or general operator handling. The limited lifetime of the connection device may be defined by a number of connections and/or a duration of use. Therefore, aspects of this disclosure are directed to medical systems and methods for monitoring usage of limited use components, such as the connection device, and performing one or more actions to indicate to an operator when the lifetime of the limited use component is determined to be approaching and/or has been reached based on the usage monitoring.

The connection device may include a first connector configured to removably connect to the computing system and a second connector configured to removably connect to the medical device. Connection and usage of the first connector and second connector may not necessarily correspond one to one. Also, each of the first connector and second connector may be associated with a different number of connections and/or a duration of use defining the lifetime due to differences in the specifications and/or requirements of the first connector and second connector. For example, the first connector and second connector may have components (e.g., dictated by the specification and/or requirements) that wear at different rates. Therefore, the usage monitoring may be performed independently for each of the first connector and second connector, and the one or more actions may be performed based on a determination that at least one of the first connector and second connector is approaching or has reached the respective number of connections and/or duration of use.

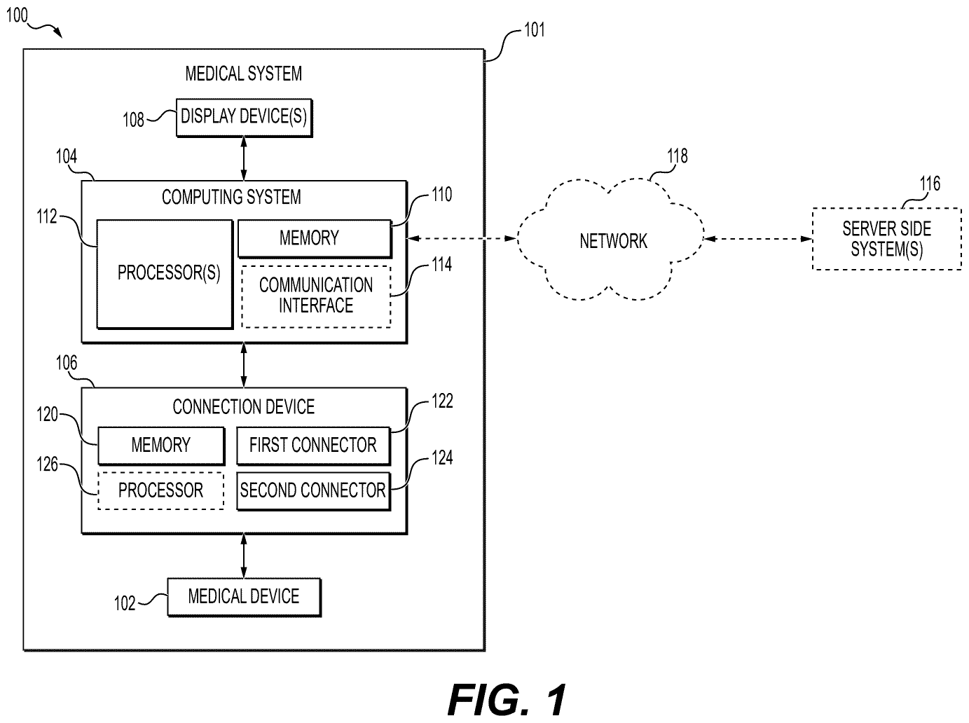

FIG. 1 depicts an exemplary environment 100 in which a usage monitoring process for a limited use component of a medical system 101 may be implemented. Environment 100 may include medical system 101, one or more optional server side system(s) 116, and/or an optional network 118 to communicatively couple medical system 101 to optional server side system(s) 116. Medical system 101 may include one or more of a medical device 102, a computing system 104, a connection device 106, and one or more display device(s) 108.

Medical device 102 may be used to perform a diagnostic and/or interventional medical procedure on a patient upon connection with computing system 104. Medical device 102 may be a single use device that is discarded or disposed of upon disconnection from computing device 102 (e.g., at an end of the medical procedure). Therefore, although only one medical device 102 is shown in FIG. 1, environment 100 may include one or more other medical devices 102 that are the same or similar to medical device 102, and may also be removably connected to computing system 104 for performing medical procedures.

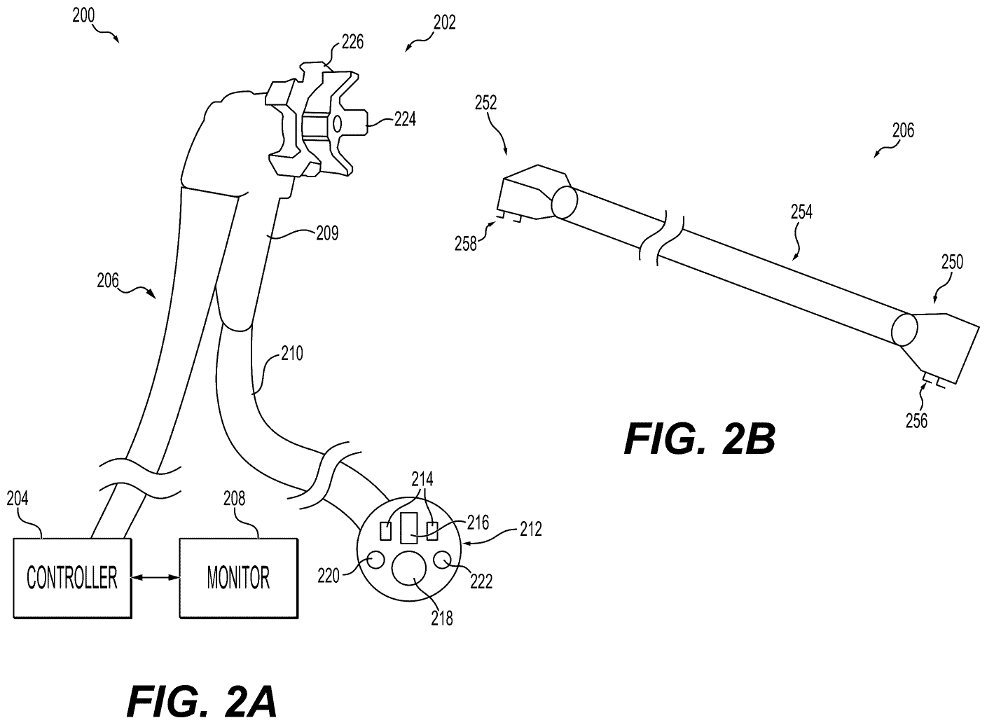

In some examples, and as described in more detail with reference to FIG. 2A, medical device 102 may be an endoscope or other type of scope or medical device having imaging capabilities, such as a cholangioscope, bronchoscope, ureteroscope, duodenoscope, gastroscope, endoscopic ultrasonography (“EUS”) scope, colonoscope, laparoscope, arthroscope, cystoscope, aspiration scope, sheath, or catheter, among other examples. In other examples, medical device 102 may include an instrument configured to perform a treatment, such as a high frequency treatment tool to cut and/or coagulate tissue, for example.

Computing system 104 may be a controller, a control unit, a computing device, or other similar standalone processing unit separate from and removably connectable to medical device 102. Computing system 104 may include a memory 110 and one or more processor(s) 112. Memory 110 may store instructions to be executed by processor(s) 112 to cause computing system 104 to perform corresponding operations. Memory 110 may also include one or more data stores. Additionally or alternatively, computing system 104 may include one or more data stores separate from memory 110. In some examples, the processor(s) 112 may be or include a field-programmable gate array (FPGA), a digital signal processing (DSP) processor, a graphics processing unit (GPU), or the like.

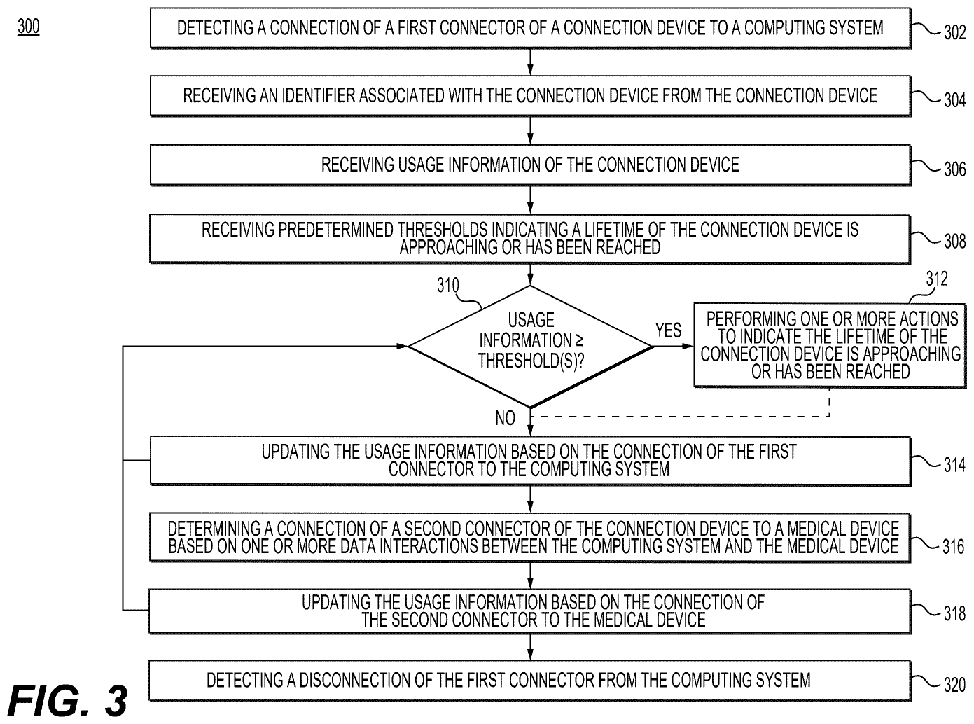

At least a portion of the instructions stored in memory 110 may include a process for monitoring usage of a limited use component of medical system 101. As described in greater detail below with reference to FIG. 3, an example usage monitoring process may include: receiving usage information associated with the limited use component and predetermined thresholds indicating that a lifetime of the limited use component is approaching or has been reached, updating the received usage information based on monitored use, comparing the received and/or updated usage information to the predetermined thresholds to determine whether the lifetime of the limited use component is approaching or has been reached, and performing one or more actions to indicate that the lifetime of the limited use component is approaching or has been reached when determined. At least one of processor(s) 112 may be configured to execute the usage monitoring process. In some examples, when medical device 102 has imaging capabilities, the instructions stored in memory 110 may also include one or more image processing operations. Additionally, processor(s) 112 may include at least one image processor configured to process, based on the stored instructions, image data captured by imaging components of medical device 102 and provided to computing system 104 to generate images.

Computing system 104 may further include an optional communication interface 114 for providing connectivity to optional network 118 to facilitate communication with optional server side system(s) 116. Although not shown in FIG. 1, optional communication interface 114 may also provide connectivity to medical device 102, connection device 106, and/or display device(s) 108. In some examples, a communicative connection between computing system 104 and medical device 102, computing system 104 and connection device 106, and/or computing system 104 and display device(s) 108 may be at least partially supported via optional network 118.

Although only one computing system 104 is shown in FIG. 1, in some examples environment 100 may include one or more other computing systems 104 that are the same or similar to computing system 104 to which connection device 106 may also be removably connected to, as described below. To provide an illustrative example, within a medical facility, multiple procedural suites may each include one computing system 104 that remains located in that suite.

Connection device 106 may be a device or device component configured to removably connect medical device 102 and computing system 104 to one another. Connection device 106 may include a memory 120, a first connector 122, a second connector 124, and/or an optional processor 126. Memory 120 may be configured to store data associated with connection device 106, including at least an identifier of connection device 106. The identifier may include a serial number or other similar information for identifying connection device 106. In some examples, a portion of the identifier may indicate a general type of connection device 106 (e.g., an umbilicus for a type of endoscope), in addition to providing information unique to the particular connection device 106 of that general type. In other examples, memory 120 may also store usage information associated with connection device 106. Memory 120 may be or include an electrically erasable programmable read-only memory (EEPROM). In some examples, memory 120 may be positioned within first connector 122 or in closer proximity to first connector 122 than second connector 124.

In some examples, and as shown and described in more detail with reference to FIGS. 2A and 2B, connection device 106 may include a tubular component (e.g., tube 254 of FIG. 2B), and first connector 122 and second connector 124 may be positioned on opposite ends of the tubular component. The tubular component may be hollow for housing any cables, wires, or other data transmitting elements extending from first connector 122 to second connector 124, to facilitate a communicative connection of computing system 104 and medical device 102. In some examples, and as discussed in more detail below, the tubular component may also house light or illumination fibers configured to transmit light for emission by medical device 102.

First connector 122 may be configured to mechanically and electrically connect connection device 106 to computing system 104. Second connector 124 may be configured to mechanically and electrically connect connection device 106 to medical device 102. First connector 122 and second connector 124 may be different connectors that are comprised of different connection components corresponding to computing system 104 and medical device 102, respectively. In other words, first connector 122 and second connector 124 include different connection components based on specifications or requirements to enable mechanical and electrical connections with computing system 104 and medical device 102, respectively.

When both first connector 122 is connected to computing system 104 and second connector 124 is connected to medical device 102, computing system 104 and medical device 102 may be enabled to communicate (e.g., data flow between computing system 104 and medical device 102 may be supported) via connection device 106. For example, data may be transmitted from computing system 104 to connection device 106 via first connector 122, from first connector 122 to second connector 124 via the cables, wires, or other data transmitting elements, and to medical device 102 via second connector 124, and vice versa. In some examples, first connector 122 and computing system 104 may have at least one exclusive electrical connection for data transmission between them (e.g., an electrical connection that does not extend through connection device 106 to medical device 102 via second connector 124).

Connection device 106 may be reusable. However, connection device 106 may have a lifetime (e.g., connection device 106 may be a limited use component or device). Connection device 106 may have components prone to wear and tear or breakage after a certain number of uses or general passage of time, which may be further impacted by sterilization and/or general operator handling. Therefore, the lifetime of connection device 106 may be defined based on cycles and/or a duration of use. Cycles may include a number of uses (e.g., a number of connections). Duration may include a total amount of usage time and/or an amount of time elapsed since an earliest connection (e.g., a first connection). In some examples, the particular number of cycles and/or duration of use for defining the lifetime may further account for impacts of sterilization and/or general operator handling.

A plurality of predetermined thresholds corresponding to or indicative of the lifetime may be utilized in the usage monitoring process, described in detail below with reference to FIG. 3. Example predetermined thresholds may include a threshold number of connections, a threshold amount of usage time, and/or a threshold amount of time elapsed since first use. In some examples, the predetermined thresholds may represent a maximum value indicating that the lifetime has been reached. In other examples, the thresholds may represent a value less than the maximum value indicating that the lifetime is approaching, but not yet reached. Because first connector 122 and second connector 124 may be different from one another for the above-discussed reasons, one or more of the predetermined thresholds may be different between, and thus specific to, first connector 122 and second connector 124. Therefore, the predetermined thresholds may include a first set and a second set of predetermined thresholds associated with first connector 122 and second connector 124, respectively, against which independently monitored usage related to first connector 122 and second connector 124 may be compared.

In some examples, connection device 106 may also include one or more electronic components that are capable of being manipulated to render connection device 106 useless. For example, initially, the electronic components may be in a closed circuit state, and serve as pass-through components to enable data flow from computing system 104 to medical device 102 via connection device 106. In response to computing system 104 generating and transmitting a trigger signal, the electronic components may be switched to an open circuit state that prevents any data flow. In some examples, and as described in more detail below, computing system 104 may generate and send the trigger signal, as part of the usage monitoring process, in response to a determination that the lifetime of connection device 106 has been reached.

In further examples, connection device 106 may include optional processor 126. Optional processor 126 may be a microcontroller, for example. In such examples, memory 120 may also be configured to store instructions for the usage monitoring process, and optional processor 126 may be configured to execute the instructions to perform the usage monitoring process. In other words, connection device 106 may perform the usage monitoring process rather than or in addition to computing system 104.

Display device(s) 108 may be configured to display data associated with one or more of the medical device 102, computing system 104, and/or connection device 106. In some examples, displayed data may include information associated with the usage and/or remaining lifetime of the limited use component (e.g., connection device 106) determined as part of the usage monitoring process. Additionally, when medical device 102 includes the imaging capabilities, the displayed data may also include images generated by computing system 104. Display device(s) 108 may include one or more a combination of monitors, computing device screens, touch screen display devices, etc. In some examples, one or more of display device(s) 108 may be a separate device from computing system 104 that is communicatively coupleable to computing system 104 via wired and/or wireless connections. In other examples, at least one of display device(s) 108 may be a display or screen of computing system 104 itself.

In some examples, computing system 104 may generate, or may cause to be generated, one or more graphical user interfaces based on instructions or information stored in memory 110, instructions or information received from one or more optional server side system(s) 116, and/or the like and may cause the graphical user interfaces to be displayed via display device(s) 108. The graphical user interfaces may include text, visual elements, controls, and/or the like, in addition to the displayed data. Display device(s) 108 may include a touch screen or a display with other input systems (e.g., a mouse, keyboard, voice, etc.) for an operator of computing system 104 to control functions of computing system 104, medical device 102 or connection device 106 via computing system 104, and/or display device(s) 108.

One or more components of environment 100, such as medical device 102, computing system 104, connection device 106, and/or display device(s) 108, may be capable of network connectivity, and may communicate with one another over a wired network or a wireless network, such as optional network 118. The network may be an electronic network. The network may include a wide area network (“WAN”), a local area network (“LAN”), personal area network (“PAN”), a cellular network (e.g., a 3G network, a 4G network, a 5G network, etc.), or the like. In other examples, the components of environment 100 may communicate and/or connect to the network over universal serial bus (USB) or other similar local, low latency connections or direct wireless protocol. Components of environment 100 may be connected via the network, using one or more standard communication protocols, such that the component may transmit and receive communications from each other across the network.

In some examples, when one or more of the components of environment 100 are capable of connecting to optional network 118, such as at least computing system 104, environment 100 may also include optional server side system(s) 116. Optional server side system(s) 116 may include one or more remote data storage systems for storing data generated by computing system 104 (e.g., data associated with the usage monitoring process and/or image data). Additionally or alternatively, when medical device 102 includes an imaging system or device, optional server side system(s) 116 may include remote image processing systems configured to perform at least a portion of the image processing, including but not limited, more resource intensive processes, such as machine learning processes (e.g., to conserve local resources of computing system 104 when network connectivity is available).

Although various components in environment 100 are depicted as separate components in FIG. 1, it should be understood that a component or portion of a component in environment 100 may, in some embodiments, be integrated with or incorporated into one or more other components. For example, one of display device(s) 108 may be integrated with computing system 104. In some embodiments, operations or aspects of one or more of the components discussed above may be distributed amongst one or more other components. Any suitable arrangement and/or integration of the various systems and devices of environment 100 may be used.

The specific examples included throughout the present disclosure implement an endoscopic imaging system configured to perform usage monitoring processes for a reusable umbilicus with a limited lifetime that is configured to removably connect the endoscope to a controller. However, it should be understood that techniques according to this disclosure may be adapted to other medical systems having a medical device that is connected to a computing system to operate via reusable cords, cables, or other similar connection devices that have a lifetime (e.g. are limited use). For example, the techniques may be adapted to a electrosurgical system comprising a high frequency (HF) generator, one or more electrodes, and/or grounding pad, where limited use connection devices may be used to connect the generator to the electrodes and/or grounding pad. It should also be understood that the examples above are illustrative only. The techniques and technologies of this disclosure may be adapted to any suitable activity.

FIG. 2A depicts an example endoscopic imaging system 200. Endoscopic imaging system 200 may be one example of medical system 101 described in detail with reference to FIG. 1. Endoscopic imaging system 200 may include a medical device 202, a controller 204, an umbilicus 206 removably connecting medical device 202 to controller 204, and a monitor 208. FIG. 2B depicts umbilicus 206 when disconnected from medical device 202 and controller 204.

Referring to FIG. 2A, medical device 202 may be one example type of medical device 102. Medical device 202 may be a single use or disposable device. Medical device 202 may be comprised of a handle 209 and a shaft 210. While medical device 202 is depicted as a cholangioscope in FIG. 2A, it will be appreciated that the disclosure also encompasses duodenoscopes, endoscopes, bronchoscopes, gastroscopes, endoscopic ultrasound (EUS) scopes, colonoscopes, ureteroscopes, bronchoscopes, laparoscopes, cystoscopes, aspiration scopes, sheaths, catheters, similar devices, or any other medical device that may be used in combination with an umbilicus or other similar cable that transmits electrical signals, power, etc.

At least a portion of shaft 210, including a distal end 212 of shaft 210, may be inserted into and navigated through a body lumen of a patient to a target site during a medical procedure, such that distal end 212 is positioned proximate to the target site. Distal end 212 may include one or more illumination devices 214, an imaging device 216, and/or distal openings for one or more of a working channel 218, a suction channel 220 and/or a fluid irrigation channel 222. Distal end 212 is shown in FIG. 2A as distal or front facing, with its components facing distally. However, in other examples, distal end 212, may be side-facing. That is, illumination devices 214, imaging device 216, and optionally one or more other distal end components, such as openings of working channel 218, suction channel 220 and/or fluid irrigation channel 222, may face radially outward, perpendicularly, approximately perpendicularly, or otherwise transverse to a longitudinal axis of the shaft 210 and distal end 212. Additionally or alternatively, distal end 212 may include one or more imaging devices 216 that face in more than one direction. For example, a first imaging device 216 may face radially outward, and a second imaging device 216 may face distally (approximately parallel to a longitudinal axis of distal end 212 and shaft 210).

Imaging device 216 may be configured to continuously capture image signals during the medical procedure as distal end 212 of medical device 102 is inserted into and navigated through the body lumen of the patient to the target site. Imaging device 216 may include one or more cameras, one or more image sensors (including analog and/or digital sensors), one or more endoscopic viewing elements, or one or more optical assemblies including one or more image sensors and one or more lenses, among other similar devices. Illumination devices 214 may be configured to receive and/or emit light to illuminate areas of the patient's body (e.g., the target site) during the medical procedure to facilitate imaging of the target site by imaging device 216. Illumination devices 214 may include one or more LEDs, incandescent light sources, optical fibers, and/or other illuminators.

A distal portion of shaft 210 that is connected to distal end 212 may have a steerable section. The steerable section may be, for example, an articulation joint. Shaft 210 and the steerable section may include a variety of structures which are known or may become known in the art. Working channel 218 may be a lumen that extends through shaft 210 and provides for delivery of instruments or treatment-related objects to the target site and/or removal of objects from the target site at distal end 212. Suction channel 220 may be a lumen that extends through shaft 210 and provides suctioning functionality at distal end 212. Fluid irrigation channel 222 may be a lumen that extends through the shaft 210 and provides irrigation functionality at distal end 212, for cleaning imaging device 216 or clearing the target site for better visualization, for example.

Handle 209 may include one or more actuators. The actuators may provide control over the steerable section, an elevator, and/or imaging functions at distal end 212 of shaft, as well as allow for the provision of air, water, suction, etc. For example, handle 209 may include control knobs 224, 226 for left, right, up, and/or down control of the steerable section of shaft 210. For example, one of control knobs 224, 226 may provide left/right control of the steerable section, and the other of control knobs 224, 226 may provide up/down control of the steerable section. Handle 209 may further include one or more locking mechanisms (e.g., knobs or levers) for preventing steering and/or braking of the steerable section in at least one of an up, down, left, or right direction. Handle 209 may include an elevator control lever (e.g., if medical device 202 is a duodenoscope). The elevator control lever may raise and/or lower an elevator, via a connection between the lever and an actuating wire that extends from the lever, through shaft 210, to the elevator.

Umbilicus 206 may be one example type of connection device 106. Umbilicus 206 removably connects to handle 209 for purposes of connecting handle 209, shaft 210, distal end 212, and components at distal end 212 to sources of, for example, power, imaging device control, image processing, light, light control, and/or display equipment. For example, umbilicus 206 may also removably connect to controller 204, and support the transmission of power and control signals from controller 204 to medical device 202, and particularly to imaging device 216 and illumination devices 214. Additionally, umbilicus 206 may support the transmission of image data from imaging device 216 to controller 204 for processing and display via monitor 208. Controller 204 may be one example type of computing system 104. Monitor 208 may be one example type of display device(s) 108.

Umbilicus 206 may connect to each of handle 209 and controller 204 via any suitable electrical and mechanical connection that will securely yet removably connect umbilicus 206 to handle 209 and controller 204, respectively, and permit the transmission of electrical signals, power, etc. between controller 204 and medical device 202 via umbilicus 206. The mechanical connection may include a mating physical connection (e.g., achieved via opposing female and male connectors). The electrical connection may include any suitable electrical connectors included in each of umbilicus 206, handle 209, and controller 204, including pins, pogo pins, card edge connectors, plugs, ports, solder connections, etc.

Turning to FIG. 2B, umbilicus 206 may include a tube 254, a controller connector 250 at a first end of tube 254, and a medical device connector 252 at a second end of tube 254 opposite of the first end. Umbilicus 206 may removably connect to controller 204 via controller connector 250 and to medical device 202 via medical device connector 252. Controller connector 250 may be one example type of first connector 122, and medical device connector 252 may be one example type of second connector 124.

Umbilicus 206 may also include electrical wires and/or optical circuitry extending within tube 254 between controller connector 250 and medical device connector 252. The electrical wires and/or optical circuity may include cables, wires, optical fibers, and/or illumination fibers for transmitting electrical signals, such as image sensor data, imager instruction signals, and power between controller 204 and medical device 202. The electrical wires and/or optical circuitry also may transmit light and/or light instruction signals from the controller to the medical device 202.

Controller connector 250 may include electrical connections 256 (e.g., in the form of connection pins or the like) for connectability to corresponding electrical connections at controller 204, which in turn electrically connect to control/power/light components and circuitry in controller 204. In some embodiments, electrical connections 256 may directly connect, through wires for example, to a circuit housed within controller connector 250. The circuit may provide image processing (for example, amplification, filtering, etc.), power, and/or electricity.

Similarly, medical device connector 252 may include electrical connections 258 (e.g., in the form of connection pins or the like) for connectability to corresponding electrical connections at handle 209 of medical device 202, which in turn may electrically connect to a circuit within handle 209. In some embodiments, electrical connections 258 may directly connect, through wires for example, to a circuit housed within medical device connector 252. These circuits may provide image processing (for example, amplification, filtering, etc.), power, and/or electricity.

During a medical procedure, imaging device 216 at distal end 212 of shaft 210 of medical device 202 may capture and transmit image data as electrical signals to handle 209. In some examples, a circuit in handle 209 may perform basic processing of the image data. The processed image data may then be transmitted as electrical signals to medical device connector 252 of umbilicus 206, via the connection between electrical connections 258 and the corresponding connections in handle 209. Alternatively, the image data may not be processed prior to transmission and/or may only be modified to, for example, amplify the electrical signals transmitted. In some examples, further processing may be performed in one or more circuits within umbilicus 206 as that image data is transmitted, via the electrical wires or optical circuitry within tube 254 extending from medical device connector 252 to controller connector 250. For example, a converter within medical device connector 252 may convert electrical signals to light signals for transmission through optical circuitry to controller connector 250. Controller connector 250 may also have a converter to convert the light signals (carrying image data, for example) back to electrical signals for transmission to controller 204. Controller 204 receives the electrical signals via controller connector 250 and processes the electrical signals (e.g., in an image processor of controller 204) to generate and cause display of an image on monitor 208, for example.

The electrical wires and/or optical circuitry extending through tube 254 may also transmit light and/or light signals and power from controller 204 to handle 209 of medical device 202. In some examples, there may be at least two pathways for the light transmission to enable a source of the light to be positioned at a different location than distal end 212 (e.g. separate from illumination devices 214). This reduces heat, and its attendant disadvantages, from distal end 212.

As one example, umbilicus 206 may include a light source, such as an LED, within a housing of medical device connector 252. In such an example, controller 204 may sends power signals and/or control signals through controller connector 250 and the optical circuitry of tube 254 to the light source within medical device connector 252. The light source may receive the signals and provide light to illumination devices 214 to cause an area surrounding distal end 212 of shaft 210 to be illuminated. For example, through the connection of the medical device connector 252 to handle 209, the light may transmit into a fiber optic cable to distal end 212 of shaft 210.

As another example, controller 204 may include the light source (e.g., LED). Controller 204 sends power signals and/or control signals to the light source within controller 204. The light source then sends light into controller connector 250 of the umbilicus 206, through the optical circuitry (e.g. fibers) within tube 254, and to medical device connector 252, where the light is transmitted via a fiber optic connector to fiber optics within handle 209 and shaft 210, to distal end 212 of medical device 202.

In use, an operator may connect umbilicus 206 to controller 204 and handle 209. The operator may then insert at least a portion of shaft 210 including distal end 212 into the body lumen of patient. Distal end 212 may be navigated to the target site. To provide visualization during navigation and at the target site, image data captured by imaging device 216 while illumination devices 214 are providing illumination may be provided to controller 204 via umbilicus 206 for processing and display on monitor 208, as described in detail above. At a conclusion of the procedure, shaft 210 may be removed from the body lumen. The operator then may disconnect umbilicus 206 from handle 209. Medical device 202 may then be disposed of. Umbilicus 206 may then be cleaned and re-used with another medical devices 202 in a subsequent procedure. In some examples, umbilicus 206 may remain connected to controller 204 while umbilicus 206 is cleaned. In other examples, umbilicus 206 may be disconnected for cleaning and connected with the same controller 204 or a different computing system similar to controller 204 for a subsequent procedure.

FIG. 3 depicts an example process 300 for monitoring usage of a limited use component of medical system 101, such as connection device 106. In some examples, one or more steps or decisions of process 300 may be performed by processor(s) 112 of computing system 104. One example type of connection device 106 for which usage may be monitored may include umbilicus 206 of endoscopic imaging system 200.

At step 302, process 300 may include detecting a connection of first connector 122 of connection device 106 to computing system 104. The connection may be an electrical and/or a mechanical connection. In some examples, first connector 122 and computing system 104 may have a dedicated or exclusive electrical connection by which computing system 104 is enabled to detect the connection.

At step 304, process 300 may include receiving an identifier associated with connection device 106 from connection device 106. For example, the connection of connection device 106 to computing system 104 may cause memory 120 of connection device 106 to become active, and transmit the identifier to computing system 104.

At step 306, process 300 may include receiving usage information of connection device 106. In some examples, the usage information may be received using the identifier, as described in detail below. The received usage information may include usage information of connection device 106 previously monitored by computing system 104 and/or one or more other computing systems 104. The received usage information may include usage information specific to each of first connector 122 and second connector 124. For example, the received usage information may include a first set of usage values associated with first connector 122. The received usage information may also include a second set of usage values associated with second connector 124.

The first and second set of usage values may be representative of previous use, if any, of connection device 106 with one or more computing systems 104 or one or more medical devices 102, respectively. Additionally, the first and second set of usage values may correspond to metrics used for defining a lifetime of connection device. For example, the lifetime of connection device 106 may be defined based on cycles and/or a duration of use. Cycles may include a number of uses (e.g., a number of connections or connection count). Duration may include a total amount of usage time and/or an amount of time elapsed from an earliest connection (e.g., first connection or use). Accordingly, the first set of usage values associated with first connector 122 may include a number of connections of first connector 122, if any, to one or more computing systems 104. The first set of usage values may also include a total amount of usage time for first connector 122 associated with the connections to one or more computing systems 104, if any, and/or an amount of time elapsed since an earliest connection of first connector 122 to one of computing systems 104. Similarly, the second set of usage values associated with second connector 124 may include a number of connections of second connector 124, if any, to one or more medical devices 102. The second set of usage values may also include a total amount of usage time for second connector 124 associated with the connections to medical devices 102, if any, and/or an amount of time elapsed since an earliest connection of second connector 124 to one of medical devices 102.

The usage information specific to first connector 122 does not necessarily, and often may not, have a one-to-one correspondence with the usage information specific to second connector 124. Therefore, the first and second set of usage values associated with first connector 122 and second connector 124, respectively, may be different from one another. To provide an example, in some medical settings, an operator may only connect connection device 106 to computing system 104 one time prior to the lifetime of connection device 106 being approached or reached. However, within that same time period, the operator may connect and disconnect connection device 106 to and from multiple medical devices 102 (e.g., cleaning connection device 106 as it remains connected to computing system 104 after the disconnection of each medical device 102). Accordingly, the first set of usage values associated with first connector 122 may have a lower connection count but higher total amount of usage time then the second set of usage values associated with second connector 124. Additionally, the time elapsed since an earliest connection may be variable between first connector 122 and second connector 124.

How the usage information is received at step 306 may be dependent on a location at which the usage information is stored. In some examples, the usage information may be stored locally on memory 110 of computing system 104. The usage information for connection device 106 may be stored in association with the identifier of connection device 106. Therefore, when the identifier is received, the identifier may be used to identify the locally stored usage information, if any, for connection device 106. If no usage information has been previously stored (e.g., connection device 106 has not been previously connected to and had usage monitored and stored by computing system 104), then a new data storage entry may be generated and associated with the identifier.

However, if the usage information is only stored locally by computing system 104, computing system 104 may be limited to storing usage information that is associated with (e.g., monitored by) computing system 104 itself. In other words, if prior to computing system 104, connection device 106 was previously connected to, used for a given amount of time, and disconnected from one or more other computing systems 104 (e.g., computing systems 104 in different procedure suites of a medical facility), usage information monitored by those other computing systems 104 would not be available to or accessible by computing system 104. Therefore, accurate usage monitoring would necessitate that connection device 106 only be connected to and used in conjunction with the same computing system 104.

Additionally or alternatively, in examples where computing system 104 is capable of network connectivity and at least one of optional server side system(s) 116 includes a remote data storage system, usage information for connection device 106 may be stored in association with the identifier of connection device 106 at the remote data storage system. Therefore, when the identifier is received, computing system 104 may generate a query using (e.g., that includes) the identifier for transmission to the data storage system (e.g., via optional communication interface 114 over optional network 118). In response, computing system 104 may receive the usage information for connection device 106. By storing the usage information remotely, any of computing systems 104 that are capable of network connectivity and have access to the remote data storage system, may be enabled to obtain the usage information. Therefore, accurate usage monitoring may be performed even when connection device 106 is connected to and used in conjunction with multiple computing systems 104.

Additionally or alternatively, the usage information may be stored locally on memory 120 of connection device 106. For example, computing system 104 may receive the usage information from connection device 106 in addition to or in lieu of the identifier upon a connection of connection device 106 to computing system 104. As described in more detail below, as connection device 106 is connected to computing system 104, updated usage information monitored by computing system 104 may be provided to and stored in memory 120 of connection device 106 in response to predefined events and/or at predefined intervals. Resultantly, upon a next connection of connection device 106 to one of computing systems 104, the usage information provided is up-to-date (e.g., includes the updated usage information). Therefore, accurate usage monitoring may be performed even when connection device 106 is connected to and used in conjunction with multiple computing systems 104.

At step 308, process 300 may include receiving predetermined thresholds indicating a lifetime of connection device 106 is approaching or has been reached. In other words, the predetermined thresholds may correspond to the metrics used for defining the lifetime of connection device 106. For example, predetermined thresholds may include a threshold number of connections, a threshold amount of usage time, and/or a threshold amount of time elapsed from an earliest connection (e.g., first connection or use). In some examples, the predetermined thresholds may represent a maximum value for a given metric indicating that the lifetime of connection device 106 has been reached. In other examples, the thresholds may represent a value less than the maximum value, indicating that the lifetime is approaching, but not yet reached.

In some examples, the predetermined thresholds may be received concurrently with and/or in a similar manner as the received usage information using the identifier, as described above in detail with reference to step 306. For example, the predetermined thresholds may be stored in association with the identifier of connection device 106 (e.g., a serial number specific to given connection device 106) or a model number general to all connection devices 106 of a same model type as given connection device 106 locally at computing system 104 and/or remotely at the data storage system. Additionally or alternatively, in some examples, a list of predetermined thresholds associated with each general type of connection device may be maintained (e.g., by computing system 104 or remote data storage system). The portion of the identifier indicating the general type of connection device 106 may be used to query the list to obtain the predetermined thresholds. Further, in other examples, the predetermined thresholds may be stored by memory 120 of connection device 106 and received therefrom upon connection to computing system 104.

Because first connector 122 and second connector 124 may have different specifications or requirements that may impact durability, for example, one or more of the predetermined thresholds may be different between, and thus specific to, first connector 122 and second connector 124. Therefore, the predetermined thresholds may include a first set of predetermined threshold values associated with first connector 122, and a second set of predetermined threshold values associated with second connector 124. For example, the first set of predetermined threshold values may include a first threshold number of connections, a first threshold amount of usage time, and/or a first threshold amount of time elapsed from an earliest connection for first connector 122. Similarly, the second set of predetermined threshold values may include a second threshold number of connections, a second threshold amount of usage time, and/or a second threshold amount of time elapsed from an earliest connections for second connector 124.

At decision 310, process 300 may include a determination of whether the usage information of connection device 106 received at step 306 meets or exceeds any of the predetermined thresholds received at step 308. Given the potential differences in both a usage of and the predetermined thresholds associated with first connector 122 and second connector 124, the determination may include a first comparison of the first set of usage values to the first set of predetermined threshold values associated with first connector 122, and a second comparison of the second set of usage values to the second set of predetermined threshold values associated with second connector 124.

For example, when performing the first comparison, a number of connections of first connector 122 to one or more computing systems 104 may be compared to the first threshold number of connections for first connector 122 to evaluate the lifetime based on cycles. Also, a total amount of usage time of first connector 122 with (e.g., a total amount of time first connector 122 has been connected to) the one or more computing systems 104 may be compared to the first threshold amount of usage time for first connector 122 to further evaluate the lifetime based on duration. Additionally or alternatively, to evaluate the lifetime based on duration, an amount of time elapsed from an earliest connection of first connector 122 to one of computing systems 104 may be compared to the first threshold amount of time elapsed from earliest connection for first connector 122.

Similarly, when performing the second comparison, a number of connections of second connector 124 to one or more medical devices 102 may be compared to the second threshold number of connections for second connector 124 to evaluate the lifetime based on cycles. Also, a total amount of usage time of second connector 124 with (e.g., a total amount of time second connector 124 has been connected to) one or more medical devices 102 may be compared to the second threshold amount of usage time for second connector 124 to further evaluate the lifetime based on duration. As described in detail with reference to step 318, usage time for second connector 124 may be based on an amount of time for which data interactions between computing systems 104 and medical devices 102 are detected. Additionally or alternatively, to evaluate the lifetime based on duration, a time elapsed from an earliest connection of second connector 124 to one of medical devices 102 may be compared to the second threshold amount of time elapsed from earliest connection for second connector 124.

The usage information of connection device 106 meeting or exceeding at least one of the predetermined thresholds, may indicate that the lifetime of the connection device is approaching or has been reached (e.g., dependent on whether the predetermined thresholds include maximum values or less than but close to maximum values). Therefore, at decision 310, if a determination is made that the usage information of connection device 106 meets or exceeds at least one of the predetermined thresholds, process 300 proceeds to step 312. For example, if at least one value from the first and/or second set of usage values meets or exceeds a corresponding value from the first and/or second set of predetermined threshold values, process 300 may proceed to step 312.

At step 312, process 300 may include performing one or more actions to indicate the lifetime of connection device 106 is approaching or has been reached. In some examples, the type of action performed may be based on whether the lifetime is approaching or whether the lifetime has been reached. Additionally, the type of action performed may be further based on a timing of the determination at decision 310, as described in detail below.

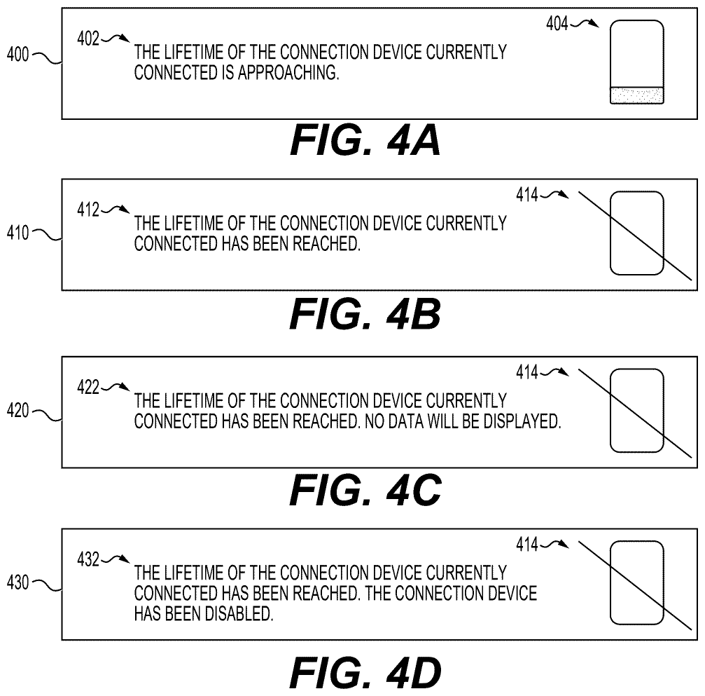

One example action performed may include generating a notification to alert the operator that the lifetime of connection device 106 is approaching or has been reached. The notification may be provided to one or more of display device(s) 108 for display. Example notifications are shown and described in detail with reference to FIGS. 4A-4D. In some examples, one or more other actions may be performed in conjunction with or as an alternative to the generation and display of the notification.

Another example action may be to render connection device 106 useless. By rendering connection device 106 useless, maintenance, expiration, and/or disposal of connection device 10 may be enforced. In some examples, connection device 106 may be rendered useless only upon a determination indicating that the lifetime of connection device 106 has been reached (as opposed to just approaching). Additionally or alternatively, in some examples, connection device 106 may only be rendered useless when the determination is made, at decision 310, upon the initial connection of first connector 122 to computing system 104 (e.g., as opposed to when the determination is made, at decision 310, at a later time after connection device 106 has been connected to and is being used in conjunction with both medical device 102 and computing system 104 during a medical procedure). This is to prevent any interruption in the medical procedure.

When medical system 101 is endoscopic imaging system 200, for example, one type of action performed to render connection device 106 useless may be for computing system 104 to stop processing and/or causing display of (e.g., via monitor 208) image data received from imaging device 216. Additionally or alternatively, when connection device 106 includes the one or more electronic components that are capable of being manipulated, another type of action performed to render connection device 106 useless may be for computing system 104 to electrically disable connection device 106. For example, initially the electronic components may be in the closed circuit state and serve as pass-through components to enable data flow from computing system 104 to medical device 102 via connection device 106. In response to the determination at decision 310, computing system 104 may generate and transmit a trigger signal to connection device 106. The trigger signal may cause the electronic components to be switched from the closed circuit state to an open circuit state that prevents any data flow.

Optionally, in some examples, process 300 may continue on to one or more of steps 314-320 discussed in detail below, as the one or more actions are being performed. To provide one example, if the action performed includes only generating and providing the notification for display (e.g., the connection device 106 is not rendered useless), then process may continue on to one or more of steps 314-320.

If, at decision 310, a determination is made that the usage information of connection device 106 does not meet or exceed at least any of the predetermined thresholds, the process proceeds to step 314. At step 314, process 300 may include updating the usage information based on the connection of first connector 122 to computing system 104. The usage information updated may be specific to first connector 122.

Updating usage information specific to first connector 122 may include incrementing a number of connections for first connector 122 by one. For example, computing system 104 may increment and store the incremented number of connections for first connector 122 based on the detected connection of first connector 122 to computing system 104 (e.g., at step 302). Updating usage information specific to first connector 122 may also include initiating a first duration counter configured to measure usage time for first connector 122. The usage time for first connector 122 may include a total amount of time first connector 122 is connected to computing system 104 from a time of detecting the connection at step 302 to a time of detecting a disconnection, as described in detail with reference to step 320 below. At predetermined time intervals (e.g., every 1 minute, every 2 minutes, etc.), the usage time indicated by the first duration counter may be stored. For example, the usage time may be added to or otherwise aggregated with any usage time previously stored in association with first connector 122 of connection device 106. For example, if the usage information received at step 306 indicates first connector 122 of connection device 106 has been used for 60 minutes, after 1 minute of measured usage by the first duration counter, the 60 minutes may be updated to 61 minutes.

One or more aspects of step 314 may be iteratively repeated (e.g., updating and storage of usage time indicated by the first duration counter at predetermined intervals). Additionally, process 300 may iteratively return to decision 310 and steps 312 and/or 314 (e.g., based on the determination made at decision 310) as first connector 122 remains connected to computing system 104. For example, the determination at decision 310 may be periodically performed based on the updated usage information, and based on the determination, process 300 may proceed to step 312, steps 312 and 314, or step 314. In some examples, the determination at decision 310 is periodically performed at the same predetermined time intervals at which the usage time indicated by the first duration counter may be stored. In other words, each time the usage information is updated with usage time, the updated usage information may be compared against the predetermined thresholds. In other examples, the determination at decision 310 may be periodically performed at different predetermined time intervals that are longer in duration than the predetermined time intervals at which the usage time indicated by the first duration counter may be stored.

At step 316, process 300 may include determining a connection of second connector 124 of connection device 106 to medical device 102 based on one or more data interactions between computing system 104 and medical device 102. In some examples, when second connector 124 is connected to medical device 102 prior to first connector 122 being connected to computing system 104, step 316 may occur concurrently with step 302. Example data interactions may include transmission (e.g., sending and/or receiving) of various types of data signals between computing system 104 and medical device 102 that indicate a complete communicative coupling or connection between computing system 104 and medical device 102. For example, when medical system 101 is endoscopic imaging system 200, example data interactions may include receiving, by controller 204, image data signals from imaging device 216, lighting signals from illumination devices 214 (e.g., indicating presence of illumination devices 214 in circuitry rather than an open circuit), and/or an indication of registration of signals sent by controller 204 to one or more components of medical device 102, such as clock signals and/or controls signals registered by imaging device 216.

At step 318, process 300 may include updating the usage information received at step 306 based on the connection of second connector 124 to medical device 102. The usage information updated may be specific to second connector 124. The usage information specific to second connector 124 may be continuously updated until a disconnection of medical device 102 from connection device 106 is detected (e.g., based on cessation of the detected data interactions), as described in detail below, and/or a disconnection of first connector 122 from computing system 104 is detected at step 320.

Updating usage information specific to second connector 124 may include incrementing a number of connections for second connector 124 by one. For example, computing system 104 may increment and store the incremented number of connections for second connector 124 based on the determined connection of second connector 124 to computing system 104 (e.g., at step 316). The specific electrical or mechanical connection of second connector 124 to medical device 102 itself may not be detected directly. However, because medical device 102 is a single use or disposable device, a new communicative or electrical connection of medical device 102 to computing system 104 via connection device 106 (e.g., detected by data interactions) assumedly corresponds to a new connection of second connector 124 to medical device 102 for which the connection number should be incremented.

Updating usage information specific to second connector 124 may also include initiating a second duration counter configured to measure usage time for second connector 124. The usage time measured may include a total amount of time for which data signals are being transmitted between computing system 104 and medical device 102. When medical system 101 is endoscopic imaging system 200, the usage time measured may specifically include an amount of time image data signals are continuously received from imaging device 216 and/or an operating time of illumination devices 214. At predetermined time intervals (e.g., every 1 minute, every 2 minutes, etc.), the usage time indicated by the second duration counter may be stored. For example, the usage time may be added to or otherwise aggregated with any usage time previously stored in association with second connector 124 of connection device 106. For example, if the usage information received at step 306 indicates second connector 124 of connection device 106 had previously been used for 20 minutes, after 1 minute of measured usage by duration counter, the 20 minutes may be updated to 21 minutes.

One or more aspects of step 318 may be iteratively repeated (e.g., updating and storage of updated usage time indicated by the second duration counter at predetermined intervals). Additionally, process 300 may iteratively return to decision 310 and steps 312, 314, 316, and/or 318 (e.g., based on the determination made at decision 310) as medical device 102 remains connected to computing system 104 via connection device 106. For example, the determination at decision 310 may be periodically performed based on the updated usage information, and based on the determination, process 300 may proceed to step 312, steps 312, 314, and 318, or steps 314 and 318.

In other examples, medical device 102 may be a first medical device that is disconnected from connection device 106, while connection device 106 remains connected to computing system 104 via first connector 122. As a result of the disconnection, no further data signals are being transmitted between computing system 104 and medical device 102. Therefore, the second duration counter may identify at least a temporary cessation in the usage resulting in at least a temporary stop in the second duration counter and the updating of the usage information at step 318. For example, at an end of the medical procedure, medical device 102 may be disconnected from second connector 124 and disposed, connection device 106 may be cleaned while remaining connected to computing system 104, and a new medical device 102 (e.g., a second medical device) may be connected via second connector 124. In such examples, steps 316 and 318 may again be performed to determine the connection to new medical device 102, and update the usage information for second connector 124 accordingly (e.g., to increment number of connections by one and resume usage time measurements by second duration counter). Multiple new medical devices similar to medical device 102 may be connected and disconnected from second connector 124, while connection device 106 remains connected to computing system 104 via first connector 122.

In further examples, a user interface may be generated and provided for display that includes an indicator of an amount of lifetime remaining for connection device 106 based on the updated usage information. The indicator may include text and/or a graphical icon or element to visually represent an amount of lifetime used thus far and/or an amount of lifetime remaining for connection device 106. For example, the user interface may be provided for display by one of device(s) 108 and updated throughout the usage monitoring process (e.g., as long as connection device 106 remains connected to computing system 104 via first connector 122). In such examples, updating of the usage information at steps 314 and/or 318 may also include the updating of the user interface. In some aspects, the amount of lifetime used thus far and/or the amount of lifetime remaining indicated by the indicator may be based on the usage information specific to the connector for which the amount of lifetime remaining is the least (e.g., having usage values closest to meeting and/or exceeding the predetermined thresholds).

At step 320, process 300 may include detecting a disconnection of first connector 122 from computing system 104. As a result of the detected disconnection, the first duration counter may be stopped and the latest usage information prior to an immediately preceding predetermined time interval may be updated and stored for first connector 122. Similarly, if the second duration had not been previously stopped (e.g., if connection device 106 was disconnected from computing system 104 prior to disconnecting from medical device 102), the second duration counter may be stopped and the latest usage information prior to an immediately preceding predetermined time interval may be updated and stored for second connector 124. In some examples, updating the latest usage information may include rounding up.

The usage information updated and stored for first connector 122 and second connector 124 may at least initially be stored locally in memory 110 of computing system 104. However, only storing the updated usage information locally at computing system 104 limits the use of connection device 106 with only one computing system 104 to enable accurate usage monitoring of connection device 106. To prevent such limitation, in some examples, when computing system 104 is capable of network connectivity, and first connector 122 is disconnected, the updated usage information may then be transmitted over optional network 118 for remote storage in a data storage system (e.g., one of optional server side system(s) 116). In other examples, the updated usage information may be transmitted to the data storage system for remote storage at the predetermined time intervals additionally or alternatively to local storage. The updated usage information may be stored in association with the identifier received at step 304. In further examples, the usage information updated and stored for first connector 122 and second connector 124 may be transmitted for storage in memory 120 of connection device 106. As described above in more detail with reference to step 306, storage of the updated usage information remotely in the data storage system (e.g., one of optional server side system(s) 116) and/or locally in memory 120 of connection device 106 may enable accurate usage monitoring to be performed even when connection device 106 is connected to and used in conjunction with multiple computing systems 104.

Process 300 has been described as being performed by processor(s) 112 of computing system 104. However, in other examples, when connection device 106 includes optional processor 126, optional processor 126 may instead be configured to perform one or more steps of process 300. In such examples, when second connector 124 is connected to medical device 102 prior to first connector 122 being connected to computing system 104, the optional processor 126 may be configured to determine the connection and update the usage information specific to second connector 124 (e.g., perform steps 316 and 318) regardless of the lack of connection of first connector 122 to computing system 104.

Accordingly, certain aspects may include performing usage monitoring processes for limited use components of medical systems. Process 300 described above is provided merely as an example, and may include additional, fewer, different, or differently arranged steps than depicted in FIG. 3.

FIGS. 4A-4D depict example notifications that may be generated and provided for display as part of usage monitoring process 300 described above with reference to step 312. In some examples, the notifications may be generated by computing system 104 and provided to one or more of display device(s) 108 that may be integrated with or separate from computing system 104.

FIG. 4A depicts a first notification 400. First notification 400 may be displayed in response to determining that the lifetime of connection device 106 is approaching, but has not yet been reached. First notification 400 may include text 402 indicating the approach. First notification 400 may also include one or more graphical icons or elements, such as first element 404, to visually represent an amount of lifetime remaining for connection device 106. In some examples, a graphical icon or element similar to first element 404 may be continuously provided for display and updated throughout the usage monitoring process (e.g., as long as connection device 106 remains connected to computing system 104 via first connector 122). For example, first element 404 may initially start out as a fully shaded object, where the shaded area decreases as the amount of lifetime remaining for connection device 106 decreases.

FIG. 4B depicts a second notification 410. Second notification 410 may be displayed in response to determining that the lifetime of connection device 106 has been reached. Second notification 410 may include text 412 indicating that the lifetime of connection device 106 has been reached. Second notification 410 may also include one or more graphical icons or elements, such as a second element 414, to visually represent that no lifetime remains for connection device 106.

In some examples, in addition to the generation and display of a notification, such as third notification 420 depicted by FIG. 4C and fourth notification 430 depicted by FIG. 4D, an action may be taken to render connection device 106 useless. For example, when the lifetime of connection device 106 has been determined to be reached, computing system 104 may be configured to stop processing and/or displaying data received from medical device 102 and/or may be configured to disable connection device 106. In such as examples, text 422 of third notification 420 and text 432 of fourth notification 430 may further indicate the type of action taken to render connection device 106 useless. Third notification 420 and fourth notification 430 may also include one or more graphical icons or elements, such as second element 414, to visually represent that no lifetime remains for connection device 106.

Each of first notification 400, second notification 410, third notification 420, and fourth notification 430 described above is provided merely as an example, and may include additional, fewer, different, or differently arranged information and/or interactive elements than depicted in FIGS. 4A-4D, respectively.

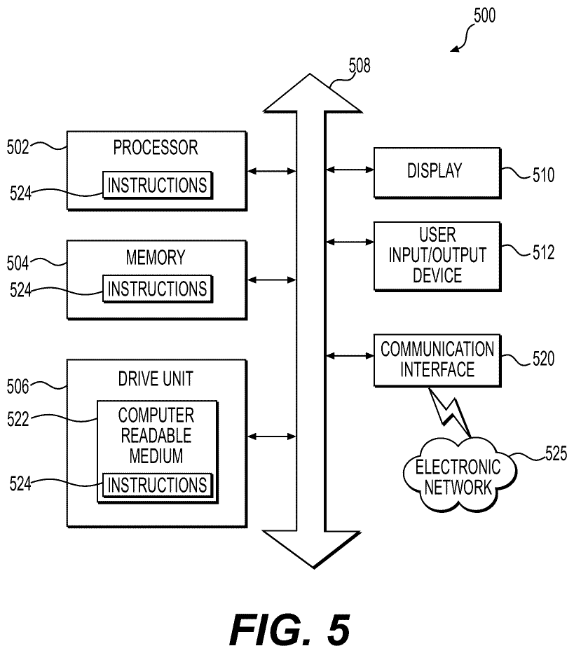

FIG. 5 depicts an example of a computer 500. FIG. 5 is a simplified functional block diagram of computer 500 that may be configured as a device for executing processes, steps, or operations depicted in, or described with respect to, FIGS. 2-4D and, according to exemplary embodiments of the present disclosure. For example, computer 500 may be configured as one or more of medical device 102, computing system 104, connection device 106, display device(s) 108, optional server side system(s) 116, and/or another device or component according to exemplary embodiments of this disclosure. In various embodiments, any of the systems herein may be or include computer 500 including, e.g., a data communication interface 520 for packet data communication. Computer 500 may communicate with one or more other computers, for example, using an electronic network 525 (e.g., via data communication interface 520). Electronic network 525 may include a wired or wireless network, for example, similar to optional network 118 depicted in FIG. 1.

Computer 500 also may include a central processing unit (“CPU”), in the form of one or more processors 502, for executing program instructions 524. In some examples, processors 502 may be or include one or more field-programmable gate arrays (FPGAs). Program instructions 524 may include at least instructions for performing usage monitoring (e.g., if computer 500 is computing system 104).

Computer 500 may include an internal communication bus 508. Computer 500 may also include a drive unit 506 (such as read-only memory (ROM), hard disk drive (HDD), solid-state disk drive (SDD), etc.) that may store data on a computer readable medium 522 (e.g., a non-transitory computer readable medium), although computer 500 may receive programming and data via network communications. Computer 500 may also have a memory 504 (such as random-access memory (RAM)) storing instructions 524 for executing techniques presented herein. It is noted, however, that in some aspects, instructions 524 may be stored temporarily or permanently within other modules of computer 500 (e.g., processor 502 and/or computer readable medium 522). Computer 500 also may include user input and output devices 512 and/or a display 510 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical system, comprising:
a computing system; and
a connection device configured to removably connect one or more medical devices to the computing system for use in one or more medical procedures, the connection device including a memory, a first connector connectable to the computing system, and a second connector, different from the first connector, connectable to the one or more medical devices, wherein the connection device has a lifetime for use;
wherein the computing system comprises at least one memory configured to store instructions and at least one processor configured to execute the instructions to perform operations to independently monitor usage of the first connector and the second connector of the connection device, the operations comprising:
detecting a connection of the first connector to the computing system;
determining a connection of the second connector to a first medical device of the one or more medical devices based on one or more data interactions detected between the computing system and the first medical device;
receiving usage information of the connection device, including usage information specific to each of the first connector and the second connector;
updating the usage information for each of the first connector and the second connector based on the connection of the first connector to the computing system and the connection of the second connector to the first medical device, respectively;
determining whether at least one of a plurality of predetermined thresholds indicating the lifetime of the connection device is approaching or has been reached has been met or exceeded based on the received usage information or the updated usage information, the plurality of predetermined thresholds including one or more predetermined thresholds specific to each of the first connector and the second connector; and
in response to determining at least one of the plurality of predetermined thresholds has been met or exceeded, performing one or more actions to indicate the lifetime of the connection device is approaching or has been reached.

2. The medical system of claim 1, the operations further comprising:
in response to the connection of the first connector to the computing system, receiving an identifier associated with the connection device stored in the memory of the connection device, wherein the identifier is used to receive one or more of the usage information of the connection device or the plurality of predetermined thresholds.

3. The medical system of claim 1, wherein the received usage information and the updated usage information is stored at least one of: locally by the computing system, locally in the memory of the connection device, or remotely by a data storage system.

4. The medical system of claim 1, wherein the received usage information includes usage information of the connection device previously monitored by the computing system or one or more other computing systems.

5. The medical system of claim 1, wherein the received usage information includes one or more of a number of connections, a total amount of usage time, or an amount of time elapsed from an earliest connection associated with each of the first connector and the second connector.

6. The medical system of claim 1, wherein the plurality of predetermined thresholds includes one or more of a threshold number of connections, a threshold amount of usage time, or a threshold amount of time elapsed from an earliest connection for each of the first connector and the second connector.

7. The medical system of claim 1, wherein, for the first connector, updating the usage information comprises:
in response to detecting the connection of the first connector to the computing system:
incrementing a number of connections for the first connector; and
initiating a first duration counter configured to measure an amount of time the first connector is connected to the computing system, wherein at predetermined intervals, the amount of time indicated by the first duration counter is used to update a total amount of usage time for the first connector.

8. The medical system of claim 1, wherein, for the second connector, updating the usage information comprises:
in response to detecting the one or more data interactions between the computing system and the first medical device:
incrementing a number of connections for the second connector; and
initiating a second duration counter configured to measure an amount of time the one or more data interactions continue to occur between the computing system and the first medical device, wherein at predetermined intervals, the amount of time indicated by the second duration counter is used to update a total amount of usage time for the second connector.

9. The medical system of claim 1, the operations further comprising:
as the first connector remains connected to the computing system, determining a disconnection of the second connector from the first medical device based on a cessation of the one or more data interactions between the computing system and the first medical device;
determining a connection of the second connector to a second medical device of the one or more medical devices based on one or more data interactions detected between the computing system and the second medical device; and
updating the usage information for the second connector further based on the connection of the second connector to the second medical device.

10. The medical system of claim 1, wherein performing the one or more actions comprises:
generating a notification indicating the lifetime of the connection device is approaching or has been reached; and
providing the notification for display on a display device associated with the computing system.

11. The medical system of claim 1, wherein performing the one or more actions comprises:
performing one or more actions to render the connection device useless.

12. The medical system of claim 11, wherein performing the one or more actions to render the connection device useless comprises:
preventing a processing and display of data received from the first medical device via the connection device.

13. The medical system of claim 11, wherein the connection device further includes one or more electronic components that, when in a closed circuit state, serve as pass-through components to enable data flow between the computing system and the first medical device via the connection device, and wherein performing the one or more actions to render the connection device useless comprises:
electrically disabling the connection device by generating and transmitting a trigger signal to the connection device, wherein the trigger signal causes the one or more electronic components of the connection device to be switched to an open circuit state that prevents any data flow.

14. The medical system of claim 1, the operations further comprising:
generating a user interface that includes an indicator of an amount of lifetime remaining for the connection device based on the updated usage information; and
providing the user interface for display on a display device associated with the computing system.

15. The medical system of claim 1, wherein the medical system is an endoscopic imaging system, and wherein the connection device is a reusable umbilicus for removably connecting one or more endoscopes to the computing system.

16. A computing system that is removably connectable to a medical device for use during a medical procedure via a connection device, the computing system comprising:
at least one memory configured to store instructions; and
at least one processor configured to execute the instructions to perform operations to independently monitor usage of a first connector and a second connector of the connection device, the operations comprising:
detecting a connection of the connection device to the computing system via the first connector;
receiving an identifier associated with the connection device from the connection device;
using the identifier, receiving:
usage information of the connection device associated with at least a number of connections and a duration of use specific to each of the first connector and the second connector; and
a plurality of predetermined thresholds associated with at least a number of connections and a duration of use that (i) indicate a lifetime of the connection device is approaching or has been reached and (ii) are specific to each of the first connector and the second connector;
determining a connection of the second connector to the medical device based on one or more data interactions detected between the computing system and the medical device;
determining whether at least one of the plurality of predetermined thresholds has been met or exceeded based on the received usage information; and
in response to determining at least one of the plurality of predetermined thresholds has been met or exceeded, generating and providing for display a notification indicating the lifetime of the connection device is approaching or has been reached.

17. The computing system of claim 16, wherein in response to determining at least one of the plurality of predetermined thresholds has been met or exceeded, the operations further comprise:
performing one or more actions to render the connection device useless.

18. The computing system of claim 16, the operations further comprising:
updating the usage information for each of the first connector and the second connector based on the connection of the first connector to the computing system and the connection of the second connector to the medical device, respectively, wherein updating the usage information comprises:
for the first connector, in response to detecting the connection of the first connector to the computing system:
incrementing a number of connections for the first connector; and
initiating a first duration counter configured to measure an amount of time the first connector is connected to the computing system, wherein at predetermined intervals, the amount of time indicated by the first duration counter is used to update a total amount of usage time for the first connector; and
for the second connector, in response to detecting the one or more data interactions between the computing system and the medical device:
incrementing a number of connections for the second connector; and
initiating a second duration counter configured to measure an amount of time the one or more data interactions continue to occur between computing system and the medical device, wherein at predetermined intervals, the amount of time indicated by the second duration counter is used to update a total amount of usage time for the second connector.

19. A method performed by a computing system that is removably connectable to a medical device for use during a medical procedure via a connection device including a first connector and a second connector, the method comprising:
detecting a connection of the first connector of the connection device to the computing system;
receiving an identifier associated with the connection device from the connection device;
using the identifier, receiving:
usage information of the connection device specific to each of the first connector and the second connector; and
a plurality of predetermined thresholds indicating a lifetime of the connection device is approaching or has been reached, including one or more predetermined thresholds specific to each of the first connector and the second connector;
updating the usage information for the first connector based on the detected connection and a usage monitoring of the first connector as the first connector remains connected to the computing system;
determining a connection of the second connector of the connection device to the medical device based one or more data interactions between the computing system and the medical device;
independently from the first connector, updating the usage information for the second connector based on the determined connection and a usage monitoring of the second connector as the second connector remains connected to the computing system;
periodically throughout a duration of the connection of at least the first connector to the computing system, determining whether at least one of the plurality of predetermined thresholds has been met or exceeded based on the received usage information or the updated usage information; and
in response to determining at least one of the plurality of predetermined thresholds has been met or exceeded, performing one or more actions to indicate the lifetime of the connection device is approaching or has been reached, including at least generating and providing for display a notification indicating the lifetime of the connection device is approaching or has been reached.

20. The method of claim 19, wherein performing the one or more actions further comprise:
performing one or more actions to render the connection device useless.
